(19) United States
(12) Patent Application Publication
(10) Pub. No.: US 2024/0112216 A1
(43) Pub. Date: Apr. 4, 2024

(54) SYSTEM, METHODS AND DEVICES FOR INTELLIGENT ANALYSIS AND PRESCRIPTION HANDLING FOR PATIENT AND HEALTHCARE IMPROVEMENT INITIATIVES

(71) Applicant: SymmetryRx, Radnor, PA (US)

(72) Inventors: Kevin Paris, Villanova, PA (US); Hal V. Walsh, Bryn Mawr, PA (US); James F. Jordan, Mt. Lebanon, PA (US)

(73) Assignee: SymmetryRx, Radnor, PA (US)

(21) Appl. No.: 18/375,551

(22) Filed: Oct. 1, 2023

Related U.S. Application Data
(60) Provisional application No. 63/412,111, filed on Sep. 30, 2022.

(51) Int. Cl. G06Q 30/0207; G06N 20/00; G06Q 10/083; G16H 20/10; G16H 40/20

(52) U.S. Cl. CPC G06Q 30/0236; G06N 20/00; G06Q 10/0838; G16H 20/10; G16H 40/20

(57) ABSTRACT

An intelligent system, method and device for providing a discount program benefit to a patient and managing patient prescription medication programs that implements intelligent evaluations to provide prescriptions to a patient which include discount benefits that are available for a physician prescribed medication for the patient during a patient consultation with a physician prescriber. The intelligent system also may generate, process, manage and store transactional data of the patient and medication program for the patient's benefit, and intelligently monitor the patient prescription along with prescriptions of others. Patient health is promoted by facilitating patient adherence to a prescribed medication, and to avoid potential effects of a disease or condition that may result from or be exacerbated by the failure to take a medication, or to adhere to a medication schedule. Intelligent sampling further is implemented to achieve the benefits.

### CROSS-REFERENCE TO RELATED APPLICATIONS

This application claims priority to U.S. Provisional Patent Application No. 63/412,111 filed Sep. 30, 2022, the complete contents of which is herein incorporated by reference.

### BACKGROUND OF THE INVENTION

### 1. Field of the Invention

The present invention relates to the field of healthcare and more particularly to personal health via prescription management, and intelligently evaluating and analyzing the patient information to manage the prescriptions, offerings and monitoring of patient health, and providing digital health literacy to patients.

### 2. Brief Description of the Related Art

Patient care involves visits to physicians, and in many instances following instructions and directives given to the patient by a physician. Often times, the physician will prescribe medications for a patient. The physician typically operates a practice that involves overseeing a number of patients. Some physicians specialize and handle types of ailments or conditions, so that their patients consist of individuals that have, or are suspected to have certain conditions, and require certain treatments or follow up for evaluation and monitoring of a predisposed symptom.

Pharmaceuticals and, in particular, prescription medications, are involved in a large number of patient treatment care and management programs. Physicians often prescribe a pharmaceutical for treating or maintaining a patient's health. The prescription must be precise, and the physician involvement is critical to making sure the prescription medication is compatible with the patient's medical history, as well as other medications that the patient may be taking. Prescription medications are known to be safe and effective when prescribed as indicated and when taken as prescribed. It has been reported and well-documented that the factors contributing to poor medication adherence by the patient include, costs, logistics, forgetfulness, and patient concordance. In many instances, upon being given a new medication, a patient will be monitored by the prescribing physician in order to determine whether the particular genetic profile of the patient causes any of the potential side effects. A trial period for a medication is desirable, since it can be used to determine whether there is a potential side effect, and the extent and severity of the side effect. From the medication side the patient impact as well as the financial cost is significant. The Centers for Disease Control and Prevention (CDCP) date indicates that there are 125,000 preventable deaths and $100 billion to $300 billion in preventable medical costs annually due to patient non-adherence.

In many instances sample medications are made available for a prescribing physician to distribute to a patient. Sample availability typically however is limited, being based upon sales rep and physician relationship, as the physician typically does not have a large array of samples. In addition, patient assistant programs also are limited, since they are based upon sales rep knowledge and physician relationship. As a result of limitations of data sets, which are incomplete, a physician and patient use trial and monitoring of the patient in order to make evaluations of whether the medication is suitable for a particular patient. This requires the further time of the physician. In addition, patients are often resist or are reluctant to pay for medications unless they know it works.

A need exists for a way to provide samples to patients that provides more options for discounts and eligibility programs that may be available to the patient, and which improves the potential for compatibility of a prescribed medication.

### SUMMARY OF THE INVENTION

Systems, methods and devices are provided for benefitting patient procurement of a medication by lowering and reducing potential costs for the patient, and managing patient prescription medication programs, using intelligent evaluations to provide prescriptions to a patient which include matching discounts or other cost lowering programs that are available for a physician prescribed medication for the patient. The transactions also may generate, process, manage and store transactional data of the patient and medication program for the patient's benefit. The inventive systems, methods and devices may further promote patient health by facilitating patient adherence to a prescribed medication treatment for the patient to realize the full benefit of the medication, as it was designed to be used, as well as to provide the benefit to the patient to lead a healthier life, and avoid potential effects of a disease or condition that may result from or be exacerbated by the failure to take a medication, or to adhere to a medication schedule. The systems, methods and devices facilitate the patient health by providing an intelligent sampling program that is designed to provide an immediate product to the patient, directly prescribed by the physician. According to preferred embodiments, the system, method and device are made available for the physician and patient when the physician is engaged in a consultation with the patient, such as, for example, in the physician's office, or when the physician is remotely examining or consulting with the patient, such as a telehealth visit.

Embodiments of the system, methods, and devices provide immediate access to medications available via drug discount programs. The system is easy to use and does not require advance registration. The system, method and devices are designed to work seamlessly with existing systems that let prescribers write prescriptions. The system, methods and devices of the invention may also utilize drug interaction, contraindication, and genetic information to prevent or minimize the potential for prescribing a medication that would cause a harmful interaction. The system, methods and devices also allow patients to receive education and support in managing their condition, which preferably is done through personal computing devices and digital software applications that may be made available to patients in conjunction with the other portions of the system, method and devices that generate offers for the patient, and provide or deliver the program sample to the patient.

The system, method and devices are designed to reach wide across a number of pharmaceutical market participants, such as pharmaceutical companies that offer a patient medicine and/or a program for treating a disease or condition. The invention is designed to increase utilization of discount programs which are complex to administer, and further complicated by ever-changing rules and requirements that each company has which make it difficult for health care providers to sort independently. The system intelligently makes available the participating medication programs to leverage the existing contractual and service infrastructure but with an added benefit of providing the patient with the information at the time the physician prescribes the pharmaceutical product. The system also may implement a digital vaccine or other digital health tools (DHT) by providing through the use of digital technologies, applications (e.g., delivered via smart-phones, tablets, and the like) that encourage patient habits, or provide interactions, or even monitoring in order to ascertain whether a patient is taking advantage of the full benefit of a program that the patient has qualified for and in which the patient is participating. The digital vaccine as used herein refers to a digital health tool, and for example, may be embodied in the form of a digital literacy application (and not merely a patient record). According to some embodiments and implementations, patient behavior, such as taking the medication as prescribed, renewing the medication when required or needed, may be encouraged by the system to provide a neurocognitive training that benefits the patient's overall health. For example, a physician may select a medication for a patient to treat a patient condition or disease. Upon selecting the medication for prescribing, the physician may be alerted that there is a discount program available (such as a free thirty-day trial). The offer therefore may be made available to the patient, and the physician may obtain the patient's consent at the time the physician prescribes the medication. Preferably, the physician makes the selection, and thereafter, the physician is advised through a screen graphic or other indication (which preferably is immediate), that the patient qualifies for discount program (e.g., voucher for a 30-day supply). The patient, instead of having to complete and submit a form, and receive a further communication (or card) from the patient health proponent company (e.g., the pharmaceutical company), and then take that to the pharmacy to have the medication filled, may complete that by providing consent when the physician is prescribing the medication. The intelligent management may generate the proper discount program or benefit for the patient, and the patient may assent to receive the discount during the consultation with the physician (office visit, examination, or the like). The system, methods and devices may implement one or more digital health tools (DHT) to facilitate subsequent capabilities for diagnosing and treating patients (e.g., including patients who qualify for and enroll in the discount program, as well as patients who may benefit from the system learning capabilities, and AI derived or generated health improvements). The DHT's implemented by or in conjunction with the prescription program for providing medications to a patient, may further enhance healthcare delivery for the individual patient. The DHT implemented may comprise one or more computing platforms, connectivity, software, and sensors for health care and related uses to follow, collect data, and/or provide information to one or more patients. The system, methods and devices preferably may implement intelligent learning that may include responsiveness through a digital platform, to sensed data or patient information. Information may be provided to a patient directly, the patient's care giver (physician or other person), or the pharmaceutical company involved in the medication or information relating to the medication and any effectiveness, side effects, observed or tested benefit, to improve responsiveness for other patients. The information may be utilized if and when made available (through consent and/or de-identification, or other permissible usage, which may vary from time to time based on regulations). The system, methods and devices also may be used to enhance healthcare by facilitating Digital Literacy, in order to generate or provide information that may be used to appraise health information from electronic sources, including the current AI engine and systems of the invention, and apply the knowledge gained to addressing or solving a health problem.

Digital Health Tools (DHT's) have the vast potential to improve the ability to diagnose and treat disease accurately and enhance health care delivery for the individual. Digital health technologies use computing platforms, connectivity, software, and sensors for health care and related uses. Digital Literacy according to the National Institutes of Health All of Us Research Program, digital health literacy is “the ability to seek, find, understand, and appraise health information from electronic sources and apply the knowledge gained to addressing or solving a health problem.” The invention promotes and integrates DHT's and digital literacy and does so in connection with the AI and learning to improve medication potential for reaching a patient in the first instance, and for medication effectiveness, as well as other benefits obtained with the system, that may be applied through associated DHT's and literacy initiatives provided by the invention.

According to some embodiments, the patient may receive interactions through a digital technology or platform on the patient user's computing device, such as a tablet or smartphone, which is based on the program in which the patient is enrolled, and the medication prescribed for the patient. The intelligent system may be programmed to learn which information to serve to the patient based on the patient prescription (or prescriptions) and patient history, and other information made available to the system which the system's artificial intelligence may determine or generate for a particular patient. The digital technology may be carried out using the information used to verify and qualify the patient for the program. In implementations where the system possesses or has access to information other than the patient's own information, the system may be configured to serve information to a patient based on not only the patient's own information but information that the system has processed and derived through the artificial intelligence applications using information relating to patients, outcomes and reported or collected data (even where a patient is not identified).

According to preferred embodiments, the system may implement intelligent technologies to learn behaviors and interactions, as well as responses and reactions of a patient to one or more particular medications, so that feedback may be provided, either specific to the patient, or anonymously, in accordance with any Health Insurance Portability and Accountability Act of 1996 (HIPPA) or other regulations, so that determinations beneficial to the patient to whom the prescribed medication may be made, as well as other patients who may be prescribed and are taking a medication (the same or other), or those patients that may be considered for the medication (or another). The intelligent system not only qualifies a patient for a discount program, but, in addition, may contribute to a database that the public can use or that can serve the public to enhance patient care through the intelligent learning. The intelligent system may process and provide the learned information to one or more public databases so that physicians, and other healthcare personnel (pharmacists, caregivers, and others), may have additional resources that can provide indications as to whether a particular patient may benefit from, or, conversely, may be harmed by, a course of treatment (e.g., including a prescribed or to be prescribed medication or treatment program). This can enhance the efficiencies of healthcare by diverting focus to treatments that are not contradicted, and avoid treatments likely to be indicated (by the intelligent system) to be ineffective or detrimental. The intelligent system is designed to place physicians practicing in rural areas on parity with those in urban populated areas, where, for example, case varieties and ailments and their respective diagnoses in the rural areas are not as abundant as those in urban locales. The intelligent system therefore not only provides capabilities for administering a discount program and qualifying patients, but also contributes to healthcare across the healthcare system. Embodiments of the system, method and devices further facilitate patient health by providing rapid patient-physician feedback that may be used to make decisions in the treatment of a patient's condition.

According to some implementations, the system may implement intelligence functions for patients or patient groups. For example, where a patient indicates residence in a particular postal code area, the system may determine whether that patient and/or area are in an underserved community. The system may provide or make available additional services to the patient, such as for example, language selections, digital vaccines or DHT's in the form of patient reminders, additional coupons, dialogues and counseling, and other services to facilitate ensuring that the patient can continue to take the medication, and potentially remove obstacles that may prevent or hinder the patient from being able to receive or obtain the prescribed mediation, or a continuing course of the medication. Some implementations also may provide the capabilities for human interaction, with follow up calls, or the potential for a patient to contact a live person for assistance (the availability of which can be determined based on the patient information, and/or other factors such as determinations made by the intelligent system, postal code designated areas, and the like).

The system, method and device further provide the physician with a number of options, as the medication solution providers may make available their information so that the system, method and device can integrate the solution provider's information and make that information available when the physician selects a medication for a patient. In conjunction with the prescription discount management program, the inventive systems, methods and devices may also separately or integrally monitor, evaluate and generate enhancements relating to outcome measures such as patient-reported outcome and functional status measures, which may include, for example, patient experience measures, care coordination measures, and measures of appropriate use of services. The intelligent system, methods and devices may drive patient health by implementing a learning model that the intelligent system tends to as a living model using, inter alia, information from following patients who are enrolled in the discount program. The intelligent system is designed to improve the quality of healthcare for patients, while at the same time, minimize burden on clinicians, improve outcomes for patients and drive value in care for the healthcare providers, industry and patients.

The system, method and device preferably include real-time integration with provider options such as vouchers and programs provided by pharmaceutical companies that are available for patients, and operates to help a patient commence a prescribed therapy as well as to stay on the prescribed therapy. The intelligent system operates based on the physician selection for a patient to establish a direct path for automatically filling new prescriptions for a patient. The system, methods and devices are designed to minimize or eliminate patient forgetfulness and cost issues, as well as how and why the medication will help the patient. A script is initiated by the physician that ships directly to the patient's home. The system, methods and devices enable the physician to generate a script (Srx) for a patient. The script (Srx) is typically for a free trial, such as for example, a 30-day prescription supply. The 30-day trial is shipped directly to the patient (via the patient's home address). Therefore, the patient does not need to make a special trip to the pharmacy, stand in a line, or run the risk of exposure to others, or risk exposing the patient to others. The system, methods, and devices may promote contributions to public health by providing additional benefits or not requiring a patient who may be ill, and even contagious, but in need of medication, to engage with others in public. This not only benefits the patient, but others, and yet still provides the medication that the patient is required to obtain. The free 30-day trial arrives at the patient's home, and therefore, removes many of the obstacles that result in the patient non-adherence, or even the initial failure of a patient to obtain the prescribed medication. It has been reported by the CDC that in the United States, about 3.8 billion prescriptions are written annually. Cutler D M, Everett W., “Thinking outside the pillbox—medication adherence as a priority for health care reform.” N Engl J Med 2010; 362:1553-5. Reports also indicate that about 20% (approximately one in five) new prescriptions are never filled, and, among those that are filled, approximately 50% are taken incorrectly, particularly with regard to timing, dosage, frequency, and duration. Osterberg L, Blaschke T., “Adherence to medication”, N Engl J Med 2005; 353:487-97. The costs for patient non-adherence to medications are not insignificant. It also has been reported that direct health care costs associated with nonadherence have grown to approximately $100-$300 billion of U.S. health care dollars spent annually. Iuga A O, McGuire M J, “Adherence and health care costs”, Risk Manag Healthc Policy 2014; 7:35-44; and Viswanathan M, Golin C E, Jones C D, et al., “Interventions to improve adherence to self-administered medications for chronic diseases in the United States: a systematic review”, Ann Intern Med 2012; 157:785-95. With the free trial there also is no out of pocket expense, thereby eliminating yet another factor that may otherwise prevent a patient from obtaining or taking the prescribed treatment. The system, methods and devices facilitate the reduction of the economic and health burdens of a number of diseases and chronic conditions.

The system, methods and devices are intelligently configurable to cover each brand, so that there is a wide array of prescriptions available and non-limiting to the physician prescriber. According to some embodiments, the system, methods and devices may be specially configured for use by a particular brand, or may be configured to address a program or programs that target or address a particular patient condition or ailment. Embodiments preferably present offers for participation in a discount program based on multiple brands or pharmaceutical providers. There are no samples to be distributed to the physician, so the control is direct from a prescription provided by the physician to the patient, but managed though the system using intelligent evaluation and analyses to qualify the patient for the free 30-day trial. Embodiments of the system also provide reminder calls and texts, and may be further enhanced by delivery of co-pay coupons available to a patient. The system, methods and devices, can deliver a co-pay coupon based on the patient insurance characteristics and payment responsibilities. All of this intelligent management is carried out in view of the patient record.

Preferred implementations of the system, methods and devices make available the proscriptions in all 50 states so that US participants are not excluded based on geographical limitations. The system, methods and devices of the invention may be implemented to support or facilitate rural healthcare programs, and, embodiments may operate in conjunction therewith. The intelligent system also is able to initiate a transfer of the prescription after the initial distribution or delivery to the patient, so that the prescription is transferred to the patient's local pharmacy or selected pharmacy. Although the physician writes the prescription and the free trial is initiated and delivered to the patient, the process continues (for medications that are to continue for more than 30 days) by the seamless integration with the pharmacy. The system takes over to coordinate the prescription script to the pharmacy. Delivery to the patient may take place from a first location, whereas subsequent prescription fulfillment can take place at the patient's local or designed pharmacy. The system, methods and devices intelligently manage prescription characteristics and logistics with the initial script that the physician has entered for the patient.

According to embodiments of the invention, the patient may be enrolled in a program for a free medication trial at the point of consultation. The system, methods and devices are designed to operate in conjunction with other systems such as an electronic prescribing system, or “e-Prescribing,” (commonly referred to as e-prescribe). The e-Prescribing healthcare providers can enter prescription information into a computer device—like a tablet, laptop, or desktop computer—and securely transmit the prescription to pharmacies using a special software program and connectivity to a transmission network. When a pharmacy receives a request, it can begin filling the medication right away. The systems, methods and devices may operate along with an e-prescribing system; however, embodiments of the system provide and generate immediate free trials for qualifying patients at the point of issuance of the prescription, typically the physician consultation, and, through the intelligent integration with a pharmaceutical provider and a distribution pharmacy, provide delivery of the free trial to the patient. The patient is therefore qualified (if eligible) to receive a free trial of a medication during the patient visit with the physician, and the prescription is filled and delivered to the patient.

The inventive system further enhances the benefits of e-prescribing, and provides further informatic metrics that can be utilized by the smart system to not only follow patients as they receive the initial medication allotment, but also subsequent activities and health based on the prescriptions, when they are filled, and other information that is available when the patient participates in the program. The system is designed to further improve health care quality and patient safety. The smart system also utilizes drug interaction information and incorporates that into the system. The capability to provide patients with medication, and determine eligibility for a patient, also functions with the safety benefits to reduce potential medication errors, as well as prevent the prescribing of a drug that would otherwise cause a harmful or potentially harmful interaction with a medicine that the patient already takes. The qualification of the patient may include rankings based on what other drugs the patient is taking, so that the patient is assigned a ranking value that the patient cannot participate, or a ranking value based on a potential drug interaction that fails to qualify the patient for the program. According to some embodiments, the rankings may be generated by the system, and may be based on information available to the system (which can include information generated and/or collected by the system, as well as information that is made available to the system from other sources). According other embodiments, the rankings may include public information provided by the pharmaceutical company about the most suitable patient for a particular benefit program and medication. For example, the system may include a compatibility/incompatibility database, which may be referenced, generated, constructed and re-constructed by the system as it operates to make determinations of patient qualifications. The intelligent system may learn potential interactions based not only on programmed, known incompatibilities between one medication and another medication or substance (which may be available through a reference such as an electronic prescription platform), but from the constituency of incompatibilities, such as types of medication or substance. The intelligent system may undertake learning through the processing of the information to exclude or provide a lower rank to a patient for medications that, although not per se listed on the incompatibility database or other information source, match prior designations of incompatibilities based on one or more features of the incompatible or designated incompatible medicine or substance, such as based on a component, functional group, type of substance. The system learns to provide a more suitable patient eligibility qualification for a patient for a pharmaceutical prescription trial program being considered. For example, a patient correlation may be learned through patients having a genetic profile exhibiting a certain reaction, which otherwise may be undetected based solely on patient medication interactions alone. Thus the intelligent system may construct and generate a phantom or virtual database that is ongoing and derives relationships based on patient prescriptions, and other information about the prescription and the patient health.

These and other advantages of the inventive system, methods and devices may be achieved with the invention.

### BRIEF DESCRIPTION OF THE DRAWING FIGURES

FIG. 1 is a schematic illustration of a diagram showing an overview of the single point system for patient eligibility and participation in prescription trial programs integration with prescription providers and pharmacies to deliver the pharmaceutical product to the patient.

FIG. 2 is a diagram of swim lanes showing an exemplary implementation for the intelligent system where a patient visit is represented through potential qualifications for delivery of the pharmaceutical.

FIG. 3 is a schematic diagram depicting an exemplary flow mechanism for patient eligibility and pharmaceutical distribution of the system, methods and devices.

FIG. 4A is a flow diagram illustrating the coverage qualification steps of the system, method and devices.

FIG. 4B are tables providing information relating to genetic codes and DNA for usage in conjunction with the system.

FIG. 5 is a flow diagram illustrating a program qualification intelligence system of the system, methods and devices.

FIG. 6 is a flow diagram illustrating sample fulfillment, e-script and drop shipping steps of the system, methods and devices.

FIG. 7 is a flow diagram illustrating steps relating to a pharmacy retention program.

FIG. 8 is a flow diagram illustrating AI for a digital vaccine in conjunction with the patient medication adherence.

FIG. 9 is a diagram showing another exemplary implementation of the intelligent system, methods and devices according to the invention for providing eligibility qualifications for patients to participate in a medication discount program.

Appendix A is s series of flow diagrams illustrating an exemplary implementation of the intelligent system, methods and devices according to the invention for providing eligibility qualifications for patients to participate in a medication discount program.

### DETAILED DESCRIPTION OF THE INVENTION

The following may be used throughout the description and though not intended to be limited to the examples and definitions set out below.

A “computer” may refer to one or more apparatus or one or more systems that are capable of accepting a structured input, processing the structured input according to prescribed rules, and producing results of the processing as output. Examples of a computer may include: a computer, a stationary or portable computer; a computer having a single processor, multiple processors, or multi-core processors, Which may operate in parallel or not in parallel; a general purpose computer; a supercomputer; a mainframe; a super mini-computer; a mini-computer; a workstation; a micro-computer; a server; a client; an interactive television; a web appliance; a telecommunications device with internee access; a hybrid combination of a computer and an interactive television; a portable computer; a tablet personal computer (PC); a personal digital assistant (PDA); a portable telephone; application-specific hardware to emulate a computer or software, such as, for example, a digital signal processor (DSP) a field-programmable gate array (FPGA), an application specific integrated circuit (ASIC), an application specific instruction-set processor (ASIP), a chip, chips, or a chip set; a system-on-chip (SoC) or a multiprocessor system-on-chip (MPSoC); an optical computer; a quantum computer; a biological computer; and an apparatus that may accept data, may process data in accordance with one or more stored software programs, may generate results, and typically may include input, output, storage, arithmetic, logic, and control units.

“Software” may refer to prescribed rules to operate a computer or a portion of a computer. Examples of software may include: code segments; instructions; applets; pre-compiled code; compiled code; interpreted code; computer programs; and programmed logic.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a memory chip; or other types of media that can store machine-readable instructions thereon.

A “computer system” may refer to a system having one or more computers, where each computer may include a computer-readable medium embodying software to operate the computer. Examples of a computer system may include: a distributed computer system for processing information via computer systems linked by a network; two or more computer systems connected together via a network for transmitting or receiving information between the computer systems; and one or more apparatuses or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those that may be made through telephone or other communication links. A network may further include hard-wired connections (e.g., coaxial cable, twisted pair, optical fiber, waveguides, etc.) or wireless connections (e.g., radio frequency waveforms, free-space optical waveforms, acoustic waveforms, satellite transmissions, etc.). Examples of a network may include: an internet, such as the Internet; an intranet; a local area network (LAN); a wide area network (WAN); and a combination of networks, such as an internet and an intranet. Exemplary networks may operate with any of a number of protocols, such as Internet protocol (IP), asynchronous transfer mode (ATM), or synchronous optical network (SONET), user datagram protocol (UDP), IEEE 802.x, etc.

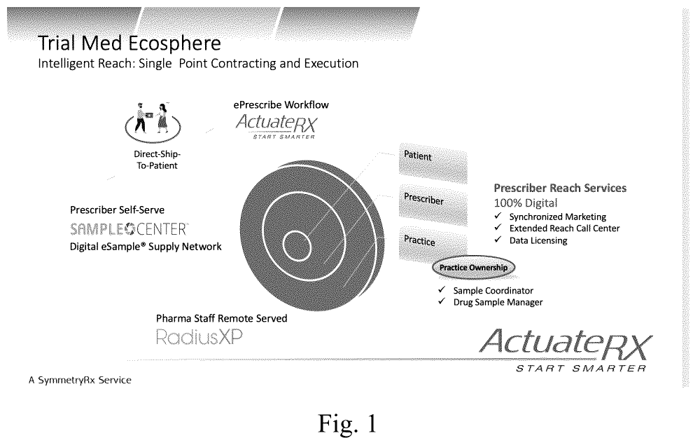

The system, method and device implement an intelligent system that reaches the physician prescriber at the point of providing the prescription. Referring to FIG. 1, a schematic diagram of a system is depicted to show some features of the inventive system. The intelligent patient sample system integrates a sample supply network with the physician and the patient to provide direct-ship-to-patient of the sample medicine that is prescribed by the patient's physician. The system, method and devices may include computers, computer systems, and data storage devices, and may operate using local, remote and/or cloud storage and applications, as well as networks. Software is provided and programmed with instructions to carry out the operations. Software also may be changed or re-programmed by the artificial intelligent (AI) functions of the system, method and devices, and databases may be generated, derived and/or revised in accordance with data received derived or generated by the AI functions of the system, method and devices.

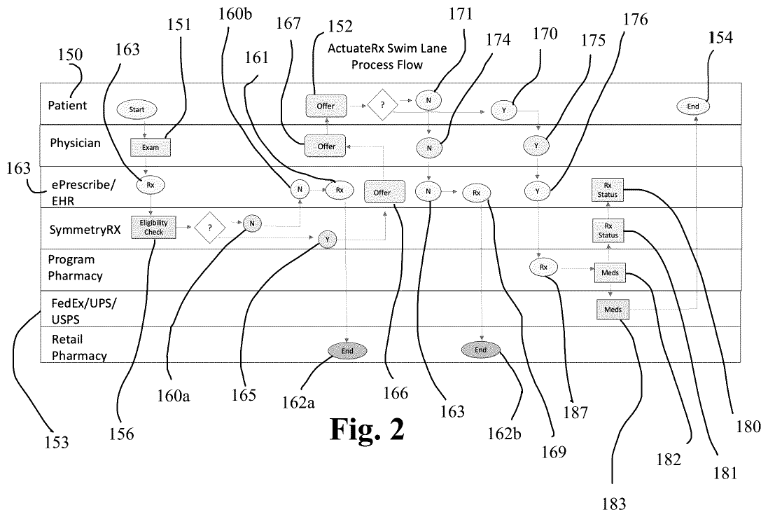

Referring to FIG. 2, there is a diagram of a swim lane process flow according to an embodiment of the invention. The swim lane diagram shows the patient lane 150 on the top row which starts with the patient visit or consultation with a physician 151. The patient lane 150 also includes one or more other actions, which in this depiction are an offer to receive a sample of the prescribed medication (sample program participation) 152, and the receipt of the sample by delivery to the patient (which in this example is via a courier 153 to the patient's address) 154. In the exemplary depiction in FIG. 2, the patient is examined by the health care provider (HCP) 151. Upon examination, if it is determined by the health care provider that the patient may benefit from or require a prescription, then the health care provider (e.g., a physician or other personnel qualified to prescribe medications) designates medication for the patient and prescribes the pharmaceutical (e.g., medication) for the patient using the patient's EHR (electronic health record) 155.

In the depiction in FIG. 2, the system, method and device establish an eligibility check 156 which coordinates any voucher program (such as a voucher for a free sample from a pharmaceutical company) with the designated prescription pharmaceutical. The system lane (SymmetryRx or SRX) 157 is intelligently automated, so that thousands or more of prescriptions may be handled in this manner, using the intelligent processing of the system. The intelligent eligibility qualifier (IEQ) takes the complexities of each pharmaceutical discount program so that the patient is not merely limited by the randomness of a particular rep that distributed a sample to the patient's physician.

Intelligent processing involves the eligibility check 156 which is able to streamline the voucher or discount process, not only for a single patient for a single medication, but for a multiplicity of patients for a number of different medications. In order to carry this out, the system is intelligently configured to utilize the patient healthcare information (which may be contained in the patient healthcare record (EHR)), and coordinate that with the voucher eligibility requirements for that patient.

The intelligent system is configured to identify the information from qualifying and non-qualifying patients to accelerate the speed and improve the functionality for subsequent prescriptions and eligibility. Intelligently generating eligibility processes through artificial intelligence and learning, facilitate streamlining an offer to a patient that will be for a medication that not only is what the prescriber requires, but also has the best chance for patient eligibility for a discount program, and compatibilities for the particular patient. For example, the intelligent system may return one or more discount programs for medications meeting the designated prescription.

Turning again to FIG. 2, when the system, upon conducting the operations and evaluations, is unable to qualify the patient for eligibility for a voucher (free trial sample) 160a, 160b, then the patient may revert to the traditional manner of obtaining the prescription, which in the current example, involves the patient obtaining a script 161 from the physician which is provided to the patient or delivered electronically to the pharmacy (via any of the commercially available eprescribing systems) 162a. This is depicted in FIG. 2, where the eligibility check lane results in a negative (N) 160a for the patient for that prescribed medication, and the prescription (Rx) 161 is filled at the pharmacy 162a. In the electronic healthcare record (EHR) of the patient, the prescription information is recorded, represented in the swim lane 163. Therefore, subsequent eligibility checks 156 for the current medication (for which the patient did not qualify for the voucher program) or for another medication (for the same or even a different ailment) may therefore include the patient having previously been prescribed with the first medication (Rx) 161. The intelligent system may utilize the prior prescription check and lack of qualification (or qualification) when conducting eligibility checks, even for the same patient for the same medication. The information also may allow pharmaceutical manufacturers and suppliers to make rules changes. The intelligent system may therefore process the rule changes, and update any actual or virtual database or information, and generate further intelligent learning that may revise qualifications or rankings (in view of any rule changes).

FIG. 2 also illustrates the situation where the patient qualifies for an eligibility program, with eligibility being indicated as affirmative or positive (Y) 165. The electronic healthcare record of the patient is updated to indicate the patient has been provided with the offer 166. In the current example, the physician is advised that the offer 166 is being provided to the patient. During the patient's consultation with the physician 151, which commenced this exemplary process illustrated in FIG. 2, the patient is examined and the physician prescribes a medication (pharmaceutical product). During the same visit or consultation, the physician prescriber is made aware of the patient qualification for the offer 167. The system, method and device provide for physician notification during the patent consultation or visit that the patient has qualified for the offer 167, and during that visit the patient may accept the offer 170 or decline the offer 171. If the patient declines the offer 171, the patient may receive the prescription 169 in the traditional route at the retail pharmacy 162b similar to the patient prescription for a patient who did not qualify for the offer (see 160a, 160b, 161, 162a). Where the patient declines the offer 171, however, the patient record (electronic healthcare record (EHR)) may be updated to indicate the patient has declined the offer 173. The physician also may be made aware of the declined offer 174. This could affect future eligibility of the patient for offers, so it is recorded.

As depicted in FIG. 2, the system, method and device are designed to receive the information about the prescription status for the patient who has accepted the offer 170. As shown in conjunction with the swim lanes. The prescription status preferably may be made available to the prescribing physician through the EHR 180, the intelligent system 181 (to provide updated information for the patient and program). The program pharmacy that fills the initial prescription 187 for the qualifying patient also has the prescription status 182, as well as the delivery status 183 (and whether the patient received the medication 154). Preferably, the status information 180, 181 also includes information about the shipment and delivery of the medication to the patient (e.g., 183, 154). The information also is maintained in the patient electronic health record (EHR) 173. The information may be used to conduct additional follow ups and provide additional coupons or discounts for a patient by estimating the medication supply delivered to the patient, and the expected renewal needs. The information also is collected, stored and processed, or otherwise made available to the intelligent system to improve qualification outcomes and rankings, as well as adjust the rankings to provide the best fit to qualify a patient for a discount program that may be in effect at the time the prescriber designates a medication for the patient. Although referred to as a physician prescriber, the system may be implemented and used with non-physician prescribers having the authority to prescribe medications for a patient (e.g., nurse practitioners, and the like).

Embodiments of the system, method and device are designed to minimize or prevent the failure of a patient to obtain and take a prescribed medication. As previously discussed, billions of dollars in cost are attributed to patient non-adherence to a prescribed medication, including the patient's failure to obtain the prescription in the first instance and/or fill the prescription. The system, method and device accelerates overcoming the first obstacle so that the patient is more likely to receive and take the medicine, and therefore, with the appreciated benefit from the first trial, the patient may therefore continue the medication. The potential for costs savings for the healthcare insurers or other payors in increased by way of preventing the worsening of a patient condition due to a lack of medication (e.g., where the patient does not fill the prescription). The system, method and device deliver a medication to the patient that is a free trial or sample of what the physician prescribed for the patient. This is handled seamlessly at the physician's office or other location where the consultation takes place (even tele- or video-medicine). The patient even may avoid having to physically visit a pharmacy, and in view of situational environments, where patients may prefer to stay at home, the present system provides the capability to do so, and does not require the patient to leave the physician's office to complete a voucher or to send or bring anything to the pharmacy.

The system, method and device communicates with a program pharmacy. Preferably, there are pharmacies that are selected or apply to join the program and are designated to participate and/or provide the patient with the trial prescription. As shown in FIG. 2, the program pharmacy lane is a designated provider which, once the patient assents to the program (by accepting the offer in the top lane), then the patient record is updated and the prescription is filled. The patient trial prescription (after the eligibility is determined) is handled by the system and through further intelligent analysis is provided to an appropriate program pharmacy. The system, method and device handle thousands of prescriptions at a time, so that once an offer is accepted, and the patient is to receive the prescribed medication, the system engages or instructs the program pharmacy 175 to provide the prescription. The delivery is then made by courier to the patient 176, as shown in the swim lane with the courier represented (which in this example is FedEx 153).

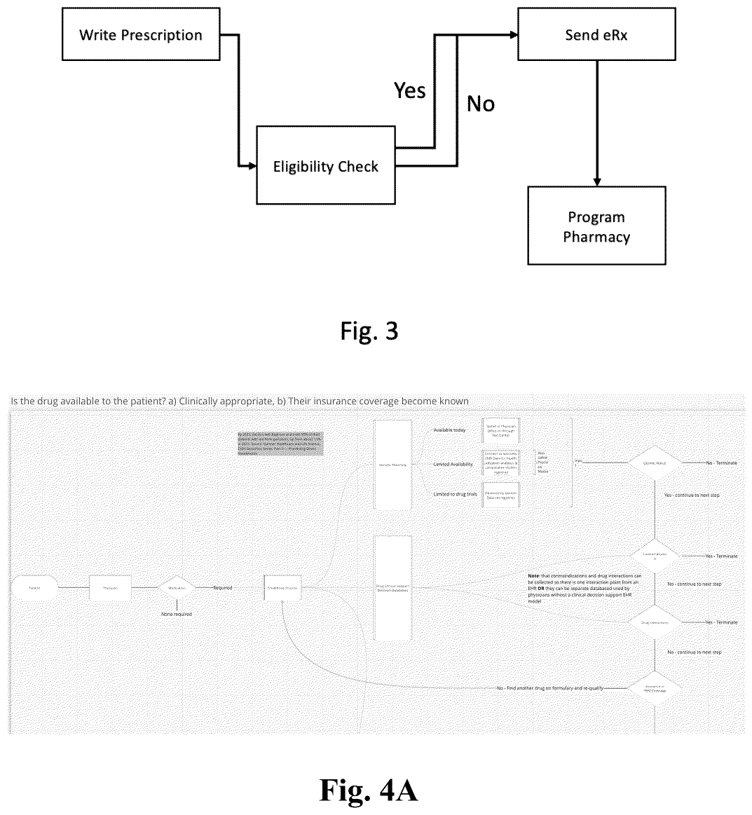

Referring to FIG. 2 and the flow diagram of FIG. 3, the system, method and device seamlessly provide the voucher program to the patient, and in a manner where the patient can receive and qualify for the voucher during the consultation with or examination by the physician prescriber. According to some preferred embodiments, as depicted in FIG. 3, the prescriber action of writing the prescription 180 (and see FIG. 2, 155) engages the engine 181 that conducts a determination for a voucher and whether the patient being prescribed that medication is eligible to receive the voucher (which may be a free trial, e.g., 30 day supply) of a medication. The determination preferably is intelligently conducted using a computer with software programmed to provide the confirmation of patient eligibility (or a denial thereof). The patient once therefore qualified receives a free trial of a medication, which is implemented during the patient visit with the physician, where the patient is qualified for eligibility in a program, and may accept the program so that the prescription is filled by a pharmacy hub and delivered to the patient.

The intelligent system is programmed and operates using an artificial intelligently programmed engine to determine patients more likely to meet eligibility for a pharmaceutical company's voucher or trial program for a prescribed medication. The engine takes the qualification away from the physician prescriber and other healthcare providers, and instead provides an intelligent ranking and, make the voucher or program available to an eligible patient when the physician prescriber has written the prescription. The intelligent engine does not require the physician to do more than make the offer to the patient. Since the physician is prescribing a medication for a patient, the system intelligently determines the ranking for patient qualification, and then presents an offer to the patient at the point of the prescriber location. The patient has the opportunity to accept or decline the offer. In either case, the physician prescribed prescription for that patient may be provided to the patient, and filled, whether in a traditional manner (without participation in the program), or through the participation in the program.

Intelligent qualification may rank patients from qualifications for the pharmaceutical product. The intelligent engine is needed to enable the presentation of multiple offers to a number of qualifying patients for different pharmaceuticals prescribed by different physicians, but at the same time. For example, a number of patients are routinely seen by physicians daily, and by qualifying patients using the patient information from the EHR and without the physician being involved for the qualification, the system, method and devices ensure an expedited process of presenting an offer, when it is available to a qualifying patient. The artificial intelligent engine may be configured to provide and rank patients, and more particularly patient qualifier items, so that the ranking for patients may be developed to identify the most likely patient to qualify, or conversely, the factors that may make a patient a more likely (higher ranked) qualifier over another. The system may intelligently determine the qualification (or qualifications) to determine first, so that the remaining qualifications, or those that are superfluous, do not consume the computing time or resources, nor do they hold up the patient or physician during the process. The intelligent system also may not necessarily use the same raking for the same duration, since the intelligent system may determine a more suitable qualifier data, and make the patient eligibility query using the more likely or then higher ranked data. Also, because the system implements artificial intelligence and learning, the patient qualifiers can be enhanced by using an analysis that may be more personalized for each patient based on factors assigned to that patient from a score analysis. The score analysis may be used to present offers to a patient that takes into account patient health, geographic location(s), compatibility with patient medications and/or conditions, patient genetics/genetic code/genetic variants, medication formulations and formulation changes, potential tolerance/intolerance for a medication, and other information that may affect one of the factors being considered in the ranking or eligibility determination.

According to some embodiments, the intelligent system in conjunction with providing an offer to a patient, may also implement a score system that enhances the healthcare value for the patient by making available further interventions or actions based on an intelligently generated or derived score. For example, where a patient is known or where the intelligent system make determinations that the patient would benefit from an enhanced service, the patient may receive additional solutions. For example, where the patient postal code indicates the patient resides in a population area that is associated with a language other than English, the patient may be provided with or provided with the option to receive materials, print and/or through the digital media and applications, in a different language. The intelligent system also may generate text in larger sized print or fonts when the system determines the patient is a particular age, or where the system finds a correlation that determines the patient data generates a score attribute such as a ranking or number, that the system intelligently derives for the patient. The intelligently derived score may provide materials in larger font when an appropriate metric is generated. The intelligent system may also generate applications for use by patients, and may determine when the application would be beneficial, such as where patient reminders on a digital application would benefit a patient, or special offers for a discount of future refills of the trial prescription.

According to some embodiments, the intelligent system may assign a reference value to one or more information pieces or elements. The ranking may be scaled, and the intelligence system may scale one or more data elements between a ranking range, so that interactions may be ranked based on an scaling e.g., from 0-20 (or other range/ranges), where, for example, the ranking scale may be coordinated with the reason or severity of the potential interaction. Where there is no reported interaction among public or publicly accessible data sets, the score may be set to a ranking designation of 20 (although he scaling could be inverse if desired). However, where the system also intelligently has generated data not only from the publicly available information, but has through patient experiences, and data available to or generated by the smart system, has learned of a potential incompatibility, the score may be adjusted, or, in addition or alternatively, the score may use the intelligently learned information, to generate a revision to the ranking system, and remove the learned incompatible medication from a potential qualifier, or rank the level at a low number, to replace, cancel, or lower the ranking number based solely on the public information (which failed to indicate a compatibility issue). Similarly, the ranking, such as for example, 0 to 20, where 20 indicates no compatibility issues, may be applied by the intelligent system based on a severity or type of potential compatibility issue. For example, where the compatibility result is only drowsiness, then the ranking may be lowered e.g., not 20, but a lower ranking, such as 15. However, where the compatibility issue involves a severe consequence, such as those that may cause disability and/or require hospitalization, such as liver failure, or abnormal heart rhythms, may be given a very low compatibility ranking (or none at all), such as 0-5. Lethal interactions, that result in fatalities would be ranked at 0, or other indication, to prevent the medication from being qualified for the patient. According to some other embodiments, the prescribing physician, pharmacies and/or others may be made aware of the intelligent system association of the learned potential adverse interactions or reactions, and the intelligent system may provide a check (as part of or independent of determining a qualification or lack thereof for the patient eligibility to participate in a discount program). The intelligent system also may derive and generate the rankings based on information it has derived and generated, or information that is made available to the system. For example, in the aforementioned example, there may be patient genetics that the system learns through the application of its intelligent learning that further make a medication compatible or incompatible. This therefore has the capability to learn, generate and then subsequently apply correlations, to qualify patients for a medication, and more particularly to intelligently qualify patients for the medications of programs that will be most suitable for the patient.

Participating EHR providers and participating eprescribing organizations are also involved in the system data for a patient. The intelligent system of the invention may rank the data to determine eligibility factors and implement ranking based on intelligent mechanisms that qualify a patient based on the most beneficial or likely qualifiers, as well as prevent or disqualify a patient where the intelligent system determines a ranking that indicates the system should fail the patient to qualify. The system therefore is able not only to provide the qualifying patient with the program eligibility and allow the patient to accept and enter the program, but also the system minimizes or eliminates risk of making an offer to a patient that may otherwise qualify where the patient could potentially have an interaction (including one listed or even one that is not listed on a medication cross-reference listing or database). The intelligent system may utilize informatics and metrics other than the listed drug interactions. For example, the intelligent system may also take into account laboratory results. In some instances, patient laboratory results and values may factor into whether a medication or change is needed to avoid potential toxicity or other adverse interactions and side effects. Even where a physician is diligent, the inventive intelligent system is designed to provide for prevention of qualifying a patient where a potential for harm due to an interaction may exist. According to some embodiments, where the information is accessible or provided, the intelligent system follows the patient pharmaceutical prescription from the trial through subsequent refills, as well as changes in medications. The intelligent system learns through rankings and correlations, and changes detected. The intelligent system is also designed to identify relationships between patients and program pharmaceuticals as well as non-program pharmaceuticals. For example, according to some embodiments, the intelligent system may use de-identified data (from a number of other patients) in order to provide a patient with an improved experience and use in qualifications to provide the best offer (if there are multiple offers), or an intelligently curated selection of offers.

The validation of a patient for eligibility to participate in the discount benefit program is undertaken through the system's intelligent operations, and, according to preferred embodiments, uses artificial intelligence to provide and manage patient qualifications, as well as other aspects of the system including patient medication adherence, and digital vaccines/digital health tools (DHT). The system, methods and devices implement machine learning and deep learning as part of an intelligent system to facilitate patient qualification for a benefit discount program to receive a prescription medicine. The system, methods and devices implement and integrate complex statistical techniques utilizing the artificial intelligence (AI) component. The AI component preferably trains itself to provide a determination of eligibility qualifications for a patient, in real time as the patient is present in the physician's office or other care facility, or during an in-person, or on-line examination. The learning or training may be based on an initial medication database that contains information about the suitability of a medication for a particular disease or other condition to be treated. The learning or training may not only utilize the database information to carry out the eligibility determinations, but also may continue to construct and build one or more additional databases that astutely qualify a patient for eligibility in a program for that patient to receive a medication. According to some embodiments, the system preferably has access to the patient record, and may for example, identify medications that the patient currently takes or is listed as being prescribed or being taken by the patient. The intelligent system may build the database by recognizing particular qualifiers as well as disqualification parameters and correlate patient data for one patient with patient data for another patient (or patients) and determine whether there are other metrics present that may provide indications in addition to, or apart from the already known or already determined indications. The AI system can learn and create profiles for eligibility as well as incompatibility or ineligibility, which can be more precise over time as the system is able to ascertain through patient information and patient qualifications for particular medications.

According to a preferred embodiment, the AI system may start with a database for the prescription medication discount program that has qualifiers that designate basic eligibility requirements for a patient to be verified for the program. The system may derive further eligibility and ineligibility qualifications based on patient information that has been determined to meet an exclusion or inclusion for the program. The system intelligently learns and generates and derives an intelligent resource, such as an intelligent database that includes the learned designation that qualifies or disqualifies a patient for eligibility for a particular medication discount program. The intelligent learning contributes to the benefit of patient health by determining whether, for a particular patient prescribed a medication, there is an eligible discount program for that patient based on the patient information, and also, based on the derived eligibility that the system has determined. The derived eligibility may be generated and derived using sources available to the system, including information for other eligibilities or ineligibilities. For example, a patient may be eligible but only of a condition is present. The patient, due to a particular patient circumstance based on the patient information for that patient, may be required to take a co-medication, supplement or other requirement. The system may derive eligibility for a patient by determining whether the patient that is required to take a co-medication, supplement or other requirement, is able to do that. This may be based on the learned derivations, where the eligibility engine includes the construction or derivation of a derivation database that also provides determinations based on the patient information for a patient, as well as learned derivations that include virtual patient data, which may be an expected, but not recorded metric for the patient. The derived virtual patient data for the patient may be used within the system, or, according to alternate embodiments, may be shared with a physician or other healthcare provider or prescriber. The shared information may be shared based on a patient input, or may be shared anonymously, as a data or metric that is recommended to investigate further.

The system, methods, and devices also are designed to track and monitor patient outcomes based on the prescription discount program enrollment, and/or the initial and/or continued course of taking the prescribed medication. For example, the system, method and devices employ intelligent learning to determine correlations between patients who have been enrolled in a discount program for a particular medication. For example, where a prescribed pharmaceutical has been reformulated by the manufacturer some patients may react fine and continue without any differences (even though there is a reformulation), while other patients may be sensitive to the reformulation (process, different ingredient, removal of an ingredient or other change). The intelligent system is able not only to track the prescriptions provided to and obtained by a patient, but also when a patient has switched a medication. The intelligent system recognizes whether the medical is a replacement for a prior medication (or medications), versus another prescription for something different. In cases where reformulations have been made by a manufacturer (e.g., scarcity of a material or changes in a process facility), typically only a physician treating a patient and the patient have knowledge of the patient reactions, such as, when the formulation does not work like the prior medication, or when there is an adverse reaction to the reformulated medication. Often the brand and labeling may remain the same or substantially identical. This information may be ascertained through the intelligent system processor based on the tracking of the patient prescriptions, including initial prescriptions, renewals and changes. It can be used to improve outcomes and efficiencies and minimize waste and time delays in arriving at a suitable and/or effective medication. The system and devices preferably includes a processing component, such as a server, computer or arrangements of components that include storage which preferably is secured storage or could-based storage. The system, method and devices are implemented using a processor and with software configured with instructions to carry out qualifications of eligibility for the patients seeking to participate or enroll in the program (such as the discount program for medication).

According to preferred embodiments, the system, methods and devices preferably are configured with a learning algorithm which can rank the qualifications for patients based on patient data, which includes already recorded patient data, as well as patient data that is based on learned outcomes derived from intelligent learning based on the patient data from one or more, or groupings of patients that are qualified or are taking a prescribed medication. The patient data preferably may be ranked or assigned a value, and because, according to some implementations, it may not exist at the time when a patient elects to participate in the discount program and is prescribed a medication, that patient in the future, as well as subsequent patients may qualify or be disqualified as a result of the intelligent learning from patient outcomes. The patient outcomes preferably are assigned a value, the algorithm intelligently learns whether in fact there is a patient outcome data element. The algorithm processes and assigns a patient outcome element where the patient has changed medications or ceased taking the medication. Preferably, the algorithm assigns a patient outcome element where the patient has replaced the medication with another medication. This information is based on identifications from the system and in conjunction with information accessible to or provided to the system from the pharmaceutical fulfillment organization, which may be the initial discount program provider of the delivered pharmaceutical (see FIG. 2), or may be a retail pharmacy that supplies the patient with another prescription. The intelligent system can assign values to the outcomes, which may not be expected, but can be identified when they arise. The values may hold a value identification unique to that value and/or another value that is a common value (such as the type of medication, category, use or other property), and a ranking and aggregation of the prevalence or lack thereof of the outcome elements. For example, if medication A is prescribed to treat a condition XX, and a patient record indicates a subsequent prescription for that patient has been generated (medication B), the system, method and devices assign one or more outcome elements and values based on medication B. For example, if medication B is also used to treat condition XX, based on the medication B common value (e.g., category, or the like) then the value would be ranked. The rankings may be derived and a database that may change and evolve based on the intelligent learning may be used to provide enhanced patient care, as well as to seek to qualify patients who are more likely to benefit from the discount program (versus those that may not benefit or could be better served by another medication). The intelligent system processes the information, and for example, aggregates the value categories for the various outcome elements and is able to therefore construct a database that can store the information such as the outcome elements and values, and learn to construct further value categories in the database based on information that is obtained from patient prescriptions, prescription management, and the patient participation in the discount program. For example, the intelligent learning system, method and devices are designed to generate learned rankings for potential patient qualifications for a discount program based on determinations from the outcome data and values (including the value categories information, as well as other value information). As an example, where a patient medication from a program is replaced in a number of patients, the system is programmed to conduct a comparison of patient data, such as a patient record or other available data in order to find a potential correlation. Where the aggregate values also coincide with another data element, such as outcome data, or patient data element from the patient data record, the system intelligently identifies and reconstructs the database with the rankings assigned to the coincident values (which may be one, or more, or a group of coincident values). The detection of a change in medications for a patient, such as a switch from medication A to medication B, or a category that medication B represents, may be used in the ranking and assigning of patient qualifications for participation or eligibility in the discount program for a particular medication.

According to some alternate embodiments, the system, method and devices may be associated to have access to patient data record that includes or comprises genetic data. This genetic data may be part of the patient record, such as a test for the presence or lack of a gene or marker, or may include genetic information from an outside source (such as a commercial genetic testing service). The intelligent system may also construct as part of the database rankings based on genetic data which may be associated with the outcome data elements. The intelligent system is designed to process the outcome data for patients participating in a discount program for a medication and provide improved effectiveness as the program is being administered. The aforementioned are examples, where a patient changes medication. However, there may be other outcome data that is positive or unexpected, where a patient stops taking a prior medication, changes dosage or frequency of the current program medication, and other outcome data. The intelligent system, methods and devices handle the data to process it to carry out learning to make an evaluation whether the outcome data is meaningful to the program or other health indicator. For example, a single patient with a changed dosage, out of a large number of patients with no changed dosage may not generate a ranking that warrants the system effecting qualifications of patients or subsequent eligibilities of patients for a program, but where say a majority of patients changed an original dosage, and then changed medications (to a different medication for treating the same condition), then the intelligent system may change patient qualifications based on the ranking generated by the change in dosage.

The rankings also may be used in conjunction with cross-platform medications, where there are multiple discount programs being offered (by different pharmaceutical companies), and one company's medication may be more suitable than another company's medication; or where a company makes multiple medications, and one of the company's medications may be more suitable for a particular patient than another of the same company's medications. However, according to preferred embodiments, the physician or other prescriber typically prescribes the medication for the patient, and then a determination of availability of a discount program is determined. However, for some selected pharmaceuticals, where the form of medication is selected based on the ingredient, and multiple medications can meet the prescribing medication substance, the intelligent system may determine the best qualifier for the patient based on the intelligent learning from the derived database of outcomes, value data and generated rankings.

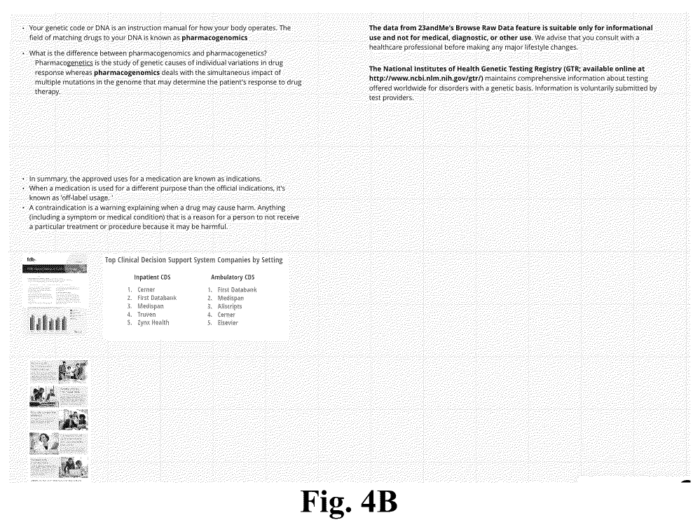

Referring to the figures the system, method and devices are depicted in implementations illustrating various functions. Referring to FIG. 4, there is illustrated preliminary checks for patient qualifications. The preliminary checks may be conducted as part of the physician determination to prescribe the medication for the patient, and according to some other embodiments, may be done additionally or alternatively, by the system. For example, there is a determination as to whether the medication is available to the patient, which may involve checks as to whether the medication is clinically appropriate, and the insurance coverage for the medication. For example, if a patient's insurance does not cover subsequent prescriptions (as may likely be needed after the first discounted medication allotment), then the determination may not consider the patient eligible (unless there is another program, discount, or consideration). If the patient is not eligible, then the system would return a negative for any programs, and the patient would receive the script from the prescribing physician or other prescriber in any conventional way. Alternatively, if the physician has multiple medications that the physician considers to be suitable or equivalent, and plans to prescribe either, the physician may commence with one, and in the event that the patient does not qualify for that one, may then commence with the other, in order for the system to determine whether the patient qualifies for that other medication.

The system, method and devices employ a number of qualifications. Qualifications may involve one or more of the following, such as genetic qualifications based on genetic matching, contraindications based on the patient health record or other information identifying a medication that the patient is taking or needs or plans to also take which is not compatible with the proposed medication for which the patient is seeking the discount program. The system, method and devices may employ access to information from the patient's electronic health record, or may access and utilize a separate database with the patient's current medication record. For example, contraindications and drug interactions may be collected so there is one interaction point from an HER, or, alternatively, they can be separate databased and used by physicians without a clinical decision support EHR model. It is anticipated by some sources that by 2025, doctors will diagnose and treat 50% of their patients with aid from genomics, up from about 1.5% in 2013. (Gartner Healthcare and Life Science CIO's Genomics Series: Part 3—Prioritizing Omics Investments). The present system, method and devices are adaptable to include genomic data for patients, as well as potential applications involving genomics, medications and combinations of therapies, as well as precision/personalized medicine models. The intelligent system may utilize genetic data specific to the patient whose eligibility for a discount program is being determined or who has enrolled (accepted the discount and program), and, according to some embodiments, may implement intelligent learning in conjunction with providing options for patient selection when offering one or more potential discounts for the patient to accept.

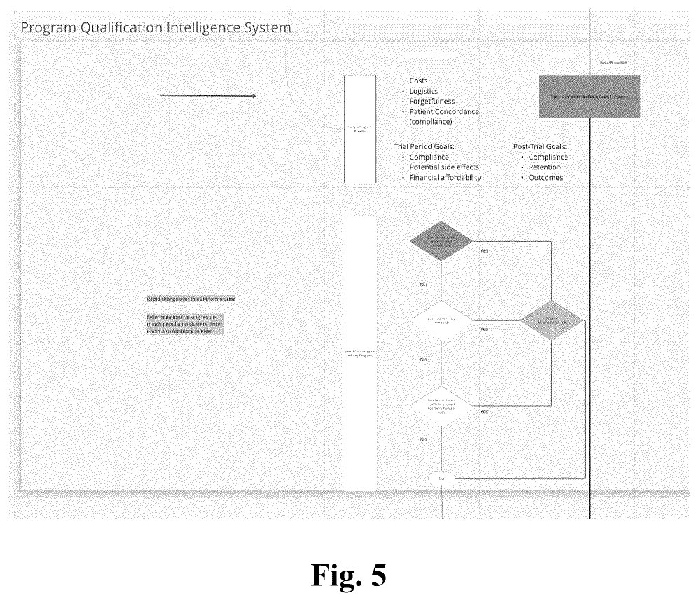

Referring to FIG. 5, the program qualification intelligence system is illustrated according to an exemplary embodiment. In FIG. 5, the qualifications may be part of the eligibility in a first stage when the physician is prescribing a medication. The diagrams illustrated in FIG. 5 may be operable to generate initial options for the physician in the first instance, based on the patient healthcare program or insurance in effect for the patient. Alternatively, the system may utilize the patient insurance qualifications in conjunction with generating an offer for the patient. For example, if a patient is eligible and receives an offer for a medication that is initially at no cost due to the program, and subsequently would not be covered for the patient, then the system may regard that patient as lacking eligibility. The system, method and devices are designed to promote patient health by providing medication that in many instances the patient can continue to take after the initial offer is accepted and the shipment is received. A benefit of the system, method and devices is to provide a medication that is prescribed for a patient that can benefit the patient, and be used to provide treatment for the patient in the future, after the initial trial has been used.

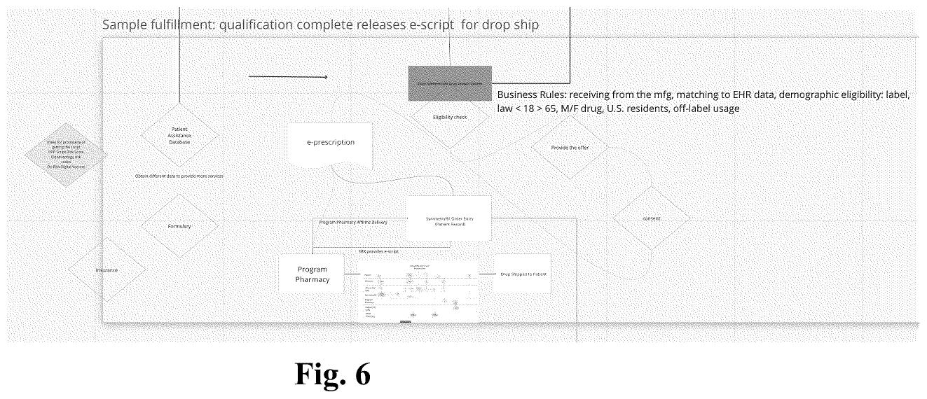

Referring to FIG. 6, when the patient has qualified for the discount program, the medication is made available to the patient, which in the depicted embodiment represented by FIG. 6, is a drop shipment of the medication directly to the patient from the program, pharmacy. The system, method and devices are designed to enhance patient healthcare by providing patients with an option to accept an offer to participate in a program to receive a no cost trial of a medication that the patient's physician or other prescriber has designated to treat the patient's condition. The intelligent system is designed to provide improved probabilities for patient care. For example, the system may use a scoring or ranking system to generate a probability script risk score for a patient. The qualification for the participation in the program may utilize the score to provide the patient with the program as well as certain program enhancements. Where a patient score for example, has a high probability for receiving the medication script, but where the intelligent system also identifies a potential risk that the patient would not refill the prescription, or regularly take the prescription, then offerings also may be made at or subsequent to the acceptance of the program, to provide further benefits to the patient. The additional benefits may include a digital vaccine or digital health tools, which may be in the form of a software application for the patient to use in order to assist the patient and the prescriber to manage the patient's care. Some examples, include reminders and logs for the patient medication, so the patient as well as the physician (if the patient desires to share the information), can determine whether the medication is effective, or whether another option is needed. For example, where the patient does not take the medication as directed, the course of action of switching medications may be avoided, since the initial medication cannot be verified as being ineffective. The intelligent system may identify patients that can benefit from additional attributes of the program, based on postal code areas, types of condition for which the medication typically it prescribed, as well as responses by other patients (which may be de-identified or anonymized data that the system processes or generates).

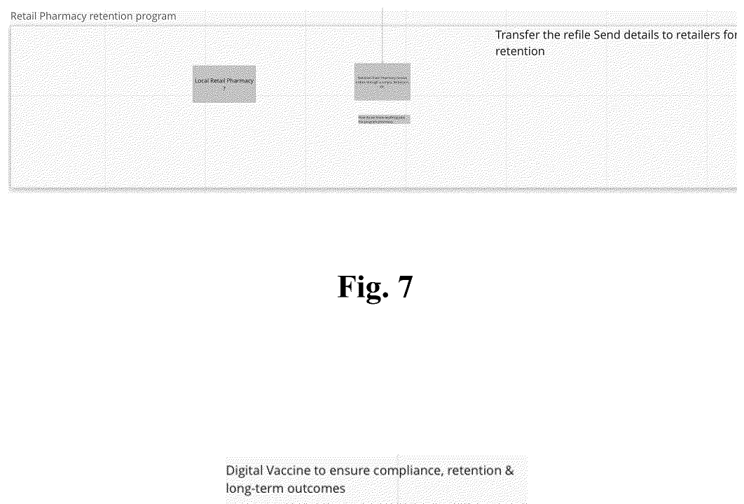

FIG. 7 illustrates the retail pharmacy potential role in retention of the continuity of the medication to make the medication available to the patient after the initial allotment from the program pharmacy. The patient record may continue to be made available to the system to determine the health benefits based on the length of time the patient maintains the prescription, as well as indications whether the patient has replaced the prescription.

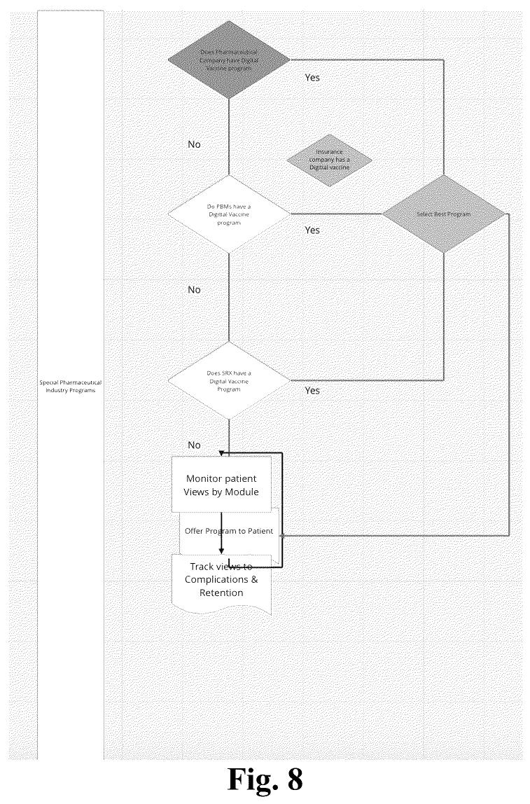

Referring to FIG. 8, there is illustrated a digital vaccine also known as a digital health tool (DHT) which is provided according to some embodiments of the system, method and devices. The digital vaccine is used to facilitate patient compliance with medications, and may be provided in the form of a digital application operable on a patient's personal computing device, such as a tablet or smartphone, and may be programmed to provide reminders to the patient. The digital vaccine or DHT may also be used in conjunction with the AI programming and processing, and enables the system to further evaluate and include data points and metrics based on patient responses with a medication. The system, method and devices may offer the digital vaccine DHT availability to a patient when the patient qualifies for the discount program. According to some alternate embodiments and implementations, the DHT may be used in conjunction with services that pharmacies may already provide (such as telephone messaging reminders), and may be used to provide a more reliable and effective enhancement or replacement to current practices.

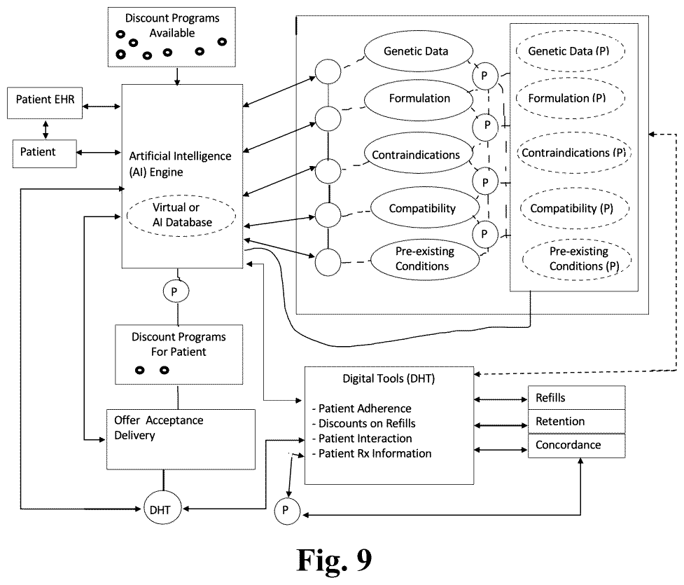

FIG. 9 is a diagram designed to illustrate an exemplary implementation of the intelligent system, method and devices according to the invention, which is designed to provide eligibility qualifications for patients to participate in a discount program to receive a trial of a medication prescribed by a prescriber. The implementations depicted, preferably includes the features of the system, methods and devices disclosed. The AI engine preferably may access the patient information and patient EHR as per the physician visit (as represented in FIG. 2). The AI engine is shown in conjunction with information elements (data) for examples of types of information that the system may utilize in order to carry out the eligibility qualification for a patient. The patient is represented by “P”. In the box to the right of the AI engine is a database containing information representing categories that include genetic information, formulation information, contraindications, compatibilities, and pre-existing conditions, although other information categories of data and information may be included. The information may be accessible to or otherwise made available to the AI engine. In the exemplary depiction, the AI engine preferably carries out learning, as discussed herein and depicted in conjunction with the other figures, to generate further data elements, which is a AI generated databased to apply to the specific patient. The AI engine may generate qualification rankings based on the patient specific information, which is shown in relation to the encircled areas “P”, and generate eligibility for the patient based on learning and using the learning to produce a virtual database for the patient which provides rankings for the patient for discount program qualifications for eligibility. If the eligibility were only simply all persons over the age of 10, that could be readily ascertained. However, the intelligent system is able to identify and determine from information made available to it, and which is accessible, as well as from patient specific information collected, observed and generated for a particular patient, to provide an eligibility ranking for a patient, that selects the discount program that has the best compatibility for the patient.

By generating a virtual database for patient data, the AI engine may learn relationships between particular patient genetic data, formulations, contraindications and other elements, to generate and revised database (or virtual database), that learns whether formulations would be suitable for a particular patient, which learning may be from other patient incompatibilities, for example through a genetic match from one or more other patients. The AI engine is shown connecting with nodes represented by open circles in the box to the right of the AI engine. The nodes show a mechanism of communications between the AI engine and information elements that may be considered for eligibility determinations. The AI engine also may be configured to manage one or more DHT's in connection with the patient healthcare and in particular the prescription management, protocols and patient services (including those depicted and described herein in connection with the DHT's). The AI system, through the utilization of digital health tools, can generate and provide reminders for patient refills, offer discounts for refills as well as provide benefits to the patient, by responding to inquiries, and providing resources for patient information, including medication information. The AI engine may administer the DHT's directly or through the façade of a pharmacy or other organization, and may seamlessly integrate as well as to collect and utilize the patient responses, feedback and identify what has been high probability in promoting patient healthcare for a patient, by promoting a response that encourages the patient to utilize the trial medication, refills, as well as to provide responses and options for patients that do not perceive a benefit, or do not appear to maintain participation in the program.

The system, methods and devices also are configured to intelligently obtain information from a qualified patient who has accepted the offer. In this manner, the pharmaceutical company may more closely follow patients, and determine the value of the benefit from the prescribed medicine. This can be done by obtaining the data when the prescription is refilled, and when to determine by this metric. However, the system also intelligently views the patient of the program as well as the other patients, and the non-program pharmaceuticals when this information also is available (as part of a patient EHR or eprescribe record). The capabilities of the system, methods and devices may be adjusted for compatibility with current health system regulations and rules, including HIPPA.

It is intended that the foregoing detailed description be regarded as illustrative rather than limiting. Numerous other changes, substitutions, variations, alterations and modifications may be ascertained by those skilled in the art and it is intended that the present invention encompass all such changes, substitutions, variations, alterations and modifications as falling within the spirit and scope of the invention and the appended claims.

What is claimed is:

1. An intelligent voucher qualifying system with immediate qualification for a patient at the point of prescriber and fulfillment of a prescribed medicine for delivery to the patient, comprising:
providing a plurality of vouchers for a plurality of patients, and for a plurality of different pharmaceutical products, for a plurality of physicians prescribing said plurality of pharmaceuticals for said plurality of patients;
intelligently obtaining patient information from a patient data source for a plurality of patients, wherein said patient data source comprises first data that includes a patient record, and second data that includes qualifying data and relationship data;
wherein said second data set is artificially intelligently manipulated to rank qualifications for patient vouchers to provide a highest ranked qualifier for a patient medication; wherein said highest ranked qualifier may include one or more data qualifiers for a patient from said patient record from said first data, and wherein said second data qualifying data is associated with relationship data to provide and rearrange ranking qualifications to improve the chances for patient qualification and expedite the process of qualifying a patient at the point of prescriber;
an engine configured to identify the current voucher qualifications, and being configured to manipulate the rankings as the patient data from said plurality of patients that qualify to present the qualifications in order to the physician prescriber with the ranking being the then current ranking that the system deems most likely to qualify for the patient.

2. The intelligent system of claim 1, wherein said engine is configured with software that includes instructions to implement learning of the qualifiers that rank together for patient qualifications to improve the workings and increase the likelihood of qualification of a patient.

3. The intelligent system of claim 1, wherein said learning is intelligently carried out for each patient prescription of a participating patient.

4. The intelligent system of claim 1, including presenting a data query based on the then current most likely to qualify rankings for patients who are qualifying for a pharmaceutical prescription program.

5. The intelligent system of claim 4, wherein said rankings include potential interactions where the ranking is effected by the potential interaction with another pharmaceutical.

6. The intelligent system of claim 5, wherein the ranking that identifies a potential interaction with another pharmaceutical is a dominant ranking, and disqualifies the patient from the program.

7. The intelligent system of claim 6, wherein when said patient is disqualified from the program, a communication is provided to the prescribing physician.

8. The intelligent system of claim 7, wherein when said communication is provided to the prescribing physician said communication indicates the circumstance for the lack of qualification of the patient.

9. The intelligent system of claim 2, including evaluating the response to the proposed most likely to qualify data query for a patient who desires eligibility in a program for a prescription, and wherein qualification of the patient is based on the then likely qualifying rank for determining eligibility.

10. The intelligent system of claim 9, including determining whether a patient ranking has a low rank that would prevent qualification.

11. The intelligent system of claim 10, wherein said low ranking is based on a potential drug interaction.

12. The intelligent system of claim 10, including qualifying the patient for participation in a pharmaceutical program for a qualifying eligible pharmaceutical product, communicating the prescription for the qualifying eligible pharmaceutical product of the program to a pharmacy that participates by co-sharing prescription information for the patient, and delivery to the patient by a courier of the qualifying eligible pharmaceutical product.

13. The intelligent system of claim 10, including a digital health software application for a personal computing device, wherein said digital health software application includes patient specific content, and receives patient inputs.

14. The intelligent system of claim 13, wherein said digital health software application provides or makes accessible the patient inputs to the software engine which comprises an artificial intelligence software engine programmed with software to implement learning, and wherein further content is delivered to the patient, and is based on intelligent learning from patient behavior which comprises learned behavior of other patients, and wherein said patient inputs further are used by the artificial intelligence software engine to implement learning and provide further patient content that is generated or determined from said learning.

15. A method for intelligently qualifying a patient to participate in a discount medication program, comprising:
providing a plurality of vouchers for a plurality of patients, and for a plurality of different pharmaceutical products, for a plurality of physicians prescribing said plurality of pharmaceuticals for said plurality of patients;
intelligently obtaining patient information from a patient data source for a plurality of patients, wherein said patient data source comprises first data that includes a patient record, and second data that includes qualifying data and relationship data;
artificially intelligently manipulating said second data set to rank qualifications for patient vouchers, and providing one or more high ranked qualifiers for a patient medication; wherein said highest ranked qualifier may include one or more data qualifiers for a patient from said patient record from said first data, and wherein said second data qualifying data is associated with relationship data to provide and rearrange ranking qualifications to improve the chances for patient qualification and expedite the process of qualifying a patient at the point of prescriber;
identifying with an artificial intelligence engine containing software configured to identify the current voucher qualifications and manipulate the rankings as the patient data from said plurality of patients that qualify, and communicating for presentation to the prescriber program offers for which the patient qualifies.
